(19) United States
(12) Patent Application Publication
(10) Pub. No.: US 2021/0219893 A1
(43) Pub. Date: Jul. 22, 2021

(54) SYSTEM AND METHOD FOR MEASUREMENT OF VOCAL BIOMARKERS OF VITALITY AND BIOLOGICAL AGING

(71) Applicant: VOCALIS HEALTH LTD., Tel Aviv (IL)

(72) Inventors: Yotam LUZ, Moshav Tal (IL); Nimrod TAIBLUM, Tel Aviv (IL); Daniella Perry ZIV, Tel Aviv (IL); Yoram LEVANON, Ramat Hasharon (IL)

(21) Appl. No.: 17/270,798

(22) PCT Filed: Aug. 26, 2019
(86) PCT No.: PCT/IL2019/050953

§ 371 Date: Feb. 23, 2021

Related U.S. Application Data
(60) Provisional application No. 62/722,918, filed on Aug. 26, 2018.

(51) Int. Cl. A61B 5/16; A61B 5/00; A61B 5/145; G10L 25/18; G10L 25/24; G10L 25/63; G10L 25/66; G10L 21/10; G16H 50/30; G16H 10/60

(52) U.S. Cl. CPC A61B 5/165; A61B 5/4803; A61B 5/7267; A61B 5/14532; A61B 5/4842; A61B 5/746; G16H 10/60; G10L 25/18; G10L 25/24; G10L 25/63; G10L 25/66; G10L 21/10; G16H 50/30; A61B 5/7282

(57) ABSTRACT

A system and method for screening and monitoring progression of subjects' health conditions and wellbeing, by the analysis of their voice signal. According to one embodiment, a system is provided that records voice samples of subjects and evaluates, in real time, the severity of their health condition based on vitality biomarkers. The vitality biomarkers are the construct of machine learning and deep learning models trained in an offline procedure. The offline training procedure is optimized to associate between (a) acoustic features and/or image representations of training cohort subjects' pre-recorded voices; and (b) their vitality score, extracted from their medical records. In the training procedure, the vitality scores of the training cohort subjects is heuristically defined as a function of the speaker age at the time of recording and the duration elapsed between the time of recording and available clinical events, with emphasis on the time of death when available.

### FIELD OF THE INVENTION

The invention is in the field of medical monitoring, and in particular for monitoring a vitality score based on voice.

### BACKGROUND TO THE INVENTION

Several systems and methods for monitoring a patient's condition based on his/her voice are previously disclosed.

U.S. Pat. No. 9,763,617B2 discloses a system and method for assessing a condition in a subject. Phones from speech of the subject are recognized, one or more prosodic or speech-excitation-source features of the phones are extracted, and an assessment of a condition of the subject, is generated based on a correlation between the features of the phones and the condition.

US20170053665A1 discloses a system and method for assessing the condition of a subject, control parameters are derived from a neurophysiological computational model that operates on features extracted from a speech signal. The control parameters are used as biomarkers (indicators) of the subject's condition. Speech related features are compared with model predicted speech features, and the error signal is used to update control parameters within the neurophysiological computational model. The updated control parameters are processed in a comparison with parameters associated with the disorder in a library.

US20120265024A1 discloses systems and methods of screening for neurological and other diseases utilizing a subject's speech behavior. According to one embodiment, a system is provided that includes an identification device used to determine a health state of a subject by receiving, as input to an interface of the device, one or more speech samples from the subject. The speech samples can be provided to the device by an intentional action of a user or passively due to the device being in the signal path of the subject's speech. The samples are communicated to a processor that identifies the acoustic measures of the samples and compares the acoustic measures of the samples with baseline acoustic measures stored in a memory of the device. The results of this determination can be communicated back to the subject or provided to a third party.

US20150265205A1 discloses detection of neurological diseases such as Parkinson's disease through analyzing a subject's speech for acoustic measures based on human factor cepstral coefficients (HFCC). Upon receiving a speech sample from a subject, a signal analysis can be performed that includes identifying articulation range and articulation rate using HFCC and delta coefficients. A likelihood of Parkinson's disease, for example, can be determined based upon the identified articulation range and articulation rate of the speech.

US20150142492A1 discloses a system that captures voice samples from a subject and determines a relative energy level of the subject from the captured voice samples. A baseline energy level for the subject is initially determined during a system training session when the subject is in a good state of health and vocalizes words or phrases for analysis by the system. Subsequently, voice samples are taken of the subject, e.g. during a work shift, to monitor the subject's fatigue levels to determine whether the subject is capable of continuing his work assignment safely, or whether the subject and the subject's work product needs to be more closely monitored. In a different application, voice samples of a subject can be taken regularly during telephone conversations, and the corresponding energy level of the subject obtained from the voice samples can be used as a general health indicator.

US20150073306A1 discloses a method of operating a computational device to process patient sounds, the method comprises the steps of: extracting features from segments of said patient sounds; and classifying the segments as cough or non-cough sounds based upon the extracted features and predetermined criteria; and presenting a diagnosis of a disease related state on a display under control of the computational device based on segments of the patient sounds classified as cough sounds.

### SUMMARY

A system and method for screening and monitoring progression of subjects' health conditions and wellbeing, by the analysis of their voice signal. According to one embodiment, a system is provided that records voice samples of subjects and evaluates, in real time, the severity of their health condition based on vitality biomarkers. The vitality biomarkers are the construct of machine learning and deep learning models trained in an offline procedure. The offline training procedure is optimized to associate between (a) acoustic features and/or image representations of training cohort subjects' pre-recorded voices; and (b) their vitality score, extracted from their medical records. In the training procedure, the vitality scores of the training cohort subjects is heuristically defined as a function of the speaker age at the time of recording and the duration elapsed between the time of recording and available clinical events, with emphasis on the time of death when available. In another embodiment, a system is provided that records subjects over time.

Analysis of repeated measurements is performed in order to evaluate progression or deterioration of diseases and pathologies and estimate risk conditions for acute events. An alert mechanism is defined, to support real-time response and trigger an appropriate treatment or other manual intervention.

It is therefore an objective of the invention to provide a computer-based system, comprising a measuring unit for estimating a vitality score of a subject based on voice and a training unit for training the measuring unit, the system comprising one or more processors and non-transitory computer-readable media (CRM), the CRMs storing instructions to the processors for operation of modules of the measuring unit 100 and the training unit,

a. the measuring unit comprising
i. one or more recording devices, configured to record a voice sample of a subject;
ii. an acoustic processing module, configured to
   a) compute temporal sequences of a set of low-level acoustic features of the voice sample; and
   b) convert the low-level sequences of acoustic features to image representations;
iii. a vocal biomarker model file, configured to store parameters of a vocal biomarker model;
iv. a vocal biomarker evaluation module, configured to evaluate a vocal biomarker of the subject as a function of the image representation, the function defined by the parameters of the vocal biomarker model; and
v. a vitality assessment module, configured to estimate a vitality score associated with the voice sample, as a function of the evaluated vocal biomarker; and

b. the training unit comprising
i. a medical records database, comprising a clinical history for subjects in a training cohort;
ii. a vitality evaluation module, configured to calculate a vitality score of each the training cohort subject, as a function of the clinical history of the training cohort subject;
iii. a voice recordings database, comprising voice clips of the training cohort subjects and their the image representations, extracted by the acoustic processing module 110; and
iv. a learning module, configured to generate the parameters of the vocal biomarker model as an optimized association of an aggregation of the vitality scores with the image representations of the training cohort and to store the vocal biomarker model in the vocal biomarker file.

It is a further objective of the invention to provide the abovementioned system, wherein the set of low-level acoustic features comprises one or more of spectrum representations, Mel-frequency cepstral coefficient (MFCC) representations, pitch and formant measures, chroma and tonal analysis, relative spectral (RASTA) analysis, linear predictive coding (LPC), line spectral pairs (LSP), perceptual linear predictive (PLP) analysis, jitter, shimmer, loudness, and any combination thereof.

It is a further objective of the invention to provide any of the abovementioned systems, wherein the learning module employs a machine-learning algorithm and generates the vocal biomarker model as a function of high-level features of the image representation; the acoustic processing module further configured to compute the high-level features.

It is a further objective of the invention to provide any of the abovementioned systems, wherein the high-level features comprise moment-analysis measurements of the low-level features, the moment analyses comprising analysis of mean, standard deviation, skewness, and kurtosis of the image representations.

It is a further objective of the invention to provide any of the abovementioned systems, wherein the learning module employs a deep learning algorithm that directly processes the image representations to generate the vocal biomarker model.

It is a further objective of the invention to provide any of the abovementioned systems, wherein the vitality score of each the training cohort subject, at a time of recording of the voice sample, is defined as a function of clinical conditions, an emotional state, physiological measurements, or any combination thereof of the training cohort subjects.

It is a further objective of the invention to provide any of the abovementioned systems, wherein the vitality score is a further a function of an age of the training cohort subject and a time duration elapsed between the time of recording and an available clinical event.

It is a further objective of the invention to provide any of the abovementioned systems, wherein the clinical events of the training cohort subjects comprise death of the subject, hospitalization of the subject, or any combination thereof.

It is a further objective of the invention to provide any of the abovementioned systems, wherein a vitality score associated with a voice clip is binary—either “0” or “1”—and “1” corresponds to “near death,” “near death” defined as when the training cohort subject died within a predefined life-end time interval or the training cohort subject exceeded a life expectancy, at a time the voice clip was recorded.

It is a further objective of the invention to provide any of the abovementioned systems, wherein the life-end interval and the life expectancy are four years and 83 years, respectively.

It is a further objective of the invention to provide any of the abovementioned systems, wherein said clinical events comprise a measurement of glycated hemoglobin (HbA1c) level.

It is a further objective of the invention to provide any of the abovementioned systems, wherein said vitality scores associated with said voice clips correspond to future HbA1c levels.

It is a further objective of the invention to provide any of the abovementioned systems, wherein the vocal biomarker model includes parameters for patterns of dynamic behavior between the features at a beginning of a voice clip and an end of the voice clip.

It is a further objective of the invention to provide any of the abovementioned systems, further comprising a personal history database configured to receive and store the evaluated vocal biomarkers to a history of the vocal biomarkers of the subject and wherein the vitality score is further a function of the history.

It is a further objective of the invention to provide any of the abovementioned systems, wherein the vocal biomarker model is further configured to evaluate, for the subject, the progression and deterioration of one or more diseases and estimate risk conditions for acute events.

It is a further objective of the invention to provide any of the abovementioned systems, wherein the voice clips and clinical events of one or more of the subjects are collected over a period of time.

It is a further objective of the invention to provide any of the abovementioned systems, wherein the diseases comprise congestive heart failure.

It is a further objective of the invention to provide any of the abovementioned systems, wherein the system is further configured to issue an alert for acute medical events of the subject.

It is a further objective of the invention to provide a computer-based process, comprising a measuring method for estimating a vitality score of a subject based on voice and a training method for training the measuring method, comprising a step of obtaining a system of claim 1, and further steps

a. of the measuring method:
i. recording a voice sample of a subject;
ii. computing temporal sequences of a set of low-level acoustic features of the voice sample;
iii. converting the low-level sequences of acoustic features to image representations;
ii. obtaining stored parameters of a vocal biomarker model;
iii. evaluating a vocal biomarker of the subject as a function of the image representation, the function defined by the parameters of the vocal biomarker model; and
iv. estimating a vitality score associated with the voice sample, as a function of the evaluated vocal biomarker; and

b. of the training method:
i. storing a clinical history for subjects in a training cohort;
ii. calculating a vitality score of each the training cohort subject, as a function of the clinical history of the training cohort subject;
iii. obtaining voice clips of the training cohort subjects and processing the voice clips in accordance with the steps of computing temporal sequences and of a set of low-level voice features and converting the low-level sequences of acoustic features to image representations;
iv. generating the parameters of the vocal biomarker model as an optimized association of an aggregation of the vitality scores with the image representations of the training cohort; and
v. storing the vocal biomarker model in a vocal biomarker file.

It is a further objective of the invention to provide the abovementioned process, wherein the set of low-level acoustic features comprises one or more of spectrum representations, Mel-frequency cepstral coefficient (MFCC) representations, pitch and formant measures, chroma and tonal analysis, relative spectral (RASTA) analysis, linear predictive coding (LPC), line spectral pairs (LSP), perceptual linear predictive (PLP) analysis, jitter, shimmer, loudness, and any combination thereof.

It is a further objective of the invention to provide any of the abovementioned processes, further comprising steps of computing high-level features of the image representation and employing a machine-learning algorithm to generate the vocal biomarker model as a function of the high-level features.

It is a further objective of the invention to provide any of the abovementioned processes, wherein the high-level features comprise moment-analysis measurements of the low-level features, the moment analyses comprising analysis of mean, standard deviation, skewness, and kurtosis of the image representations.

It is a further objective of the invention to provide any of the abovementioned processes, further comprising a step of employing a deep learning algorithm that directly processes the image representations to generate the vocal biomarker model.

It is a further objective of the invention to provide any of the abovementioned processes, wherein the vitality score of each the training cohort subject, at a time of recording of the voice sample, is defined as a function of clinical conditions, an emotional state, physiological measurements, or any combination thereof of the training cohort subjects.

It is a further objective of the invention to provide any of the abovementioned processes, wherein the vitality score is a further a function of an age of the training cohort subject and a time duration elapsed between the time of recording and an available clinical event.

It is a further objective of the invention to provide any of the abovementioned processes, wherein the clinical events of the training cohort subjects comprise death of the subject, hospitalization of the subject, or any combination thereof.

It is a further objective of the invention to provide any of the abovementioned processes, wherein a vitality score associated with a voice clip is binary—either “0” or “1”—and “1” corresponds to “near death,” “near death” defined as when the training cohort subject died within a predefined life-end time interval or the training cohort subject exceeded a life expectancy, at a time the voice clip was recorded.

It is a further objective of the invention to provide any of the abovementioned processes, wherein the life-end interval and the life expectancy are four years and 83 years, respectively.

It is a further objective of the invention to provide any of the abovementioned processes, wherein said clinical events comprise a measurement of glycated hemoglobin (HbA1c) level.

It is a further objective of the invention to provide any of the abovementioned processes, wherein said vitality scores associated with said voice clips correspond to future HbA1c levels.

It is a further objective of the invention to provide any of the abovementioned processes, wherein the vocal biomarker model includes parameters for patterns of dynamic behavior between the features at a beginning of a voice clip and an end of the voice clip.

It is a further objective of the invention to provide any of the abovementioned processes, further comprising steps of receiving and storing the evaluated vocal biomarkers to a history of the vocal biomarkers of the subject, wherein the vitality score is further a function of the history.

It is a further objective of the invention to provide any of the abovementioned processes, further comprising steps of evaluating, for the subject, the progression and deterioration of one or more diseases and estimating risk conditions for acute events.

It is a further objective of the invention to provide any of the abovementioned processes, wherein the voice clips and clinical events of one or more of the subjects are collected over a period of time.

It is a further objective of the invention to provide any of the abovementioned processes, wherein the diseases comprise congestive heart failure.

It is a further objective of the invention to provide any of the abovementioned processes, further comprising a step of issuing an alert for acute medical events of the subject.

### BRIEF DESCRIPTION OF THE DRAWINGS

FIG. 1 shows a measuring unit of a computer-based system for estimating a vitality score of a subject, according to some embodiments of the invention.

FIG. 2 shows a training unit of a computer-based system for estimating a vitality score of a subject, according to some embodiments of the invention.

FIG. 3 shows a computer-based process, comprising steps of a measuring method for estimating a vitality score of a subject and steps of a training method for training the measuring method, according to some embodiments of the invention.

### DETAILED DESCRIPTION

A paper entitled “Vocal biomarker predicts long term survival among heart failure patients,” by E. Maor et al., published in European Heart Journal, 28 Aug. 2018, page 876, is incorporated by reference in its entirety in this application.

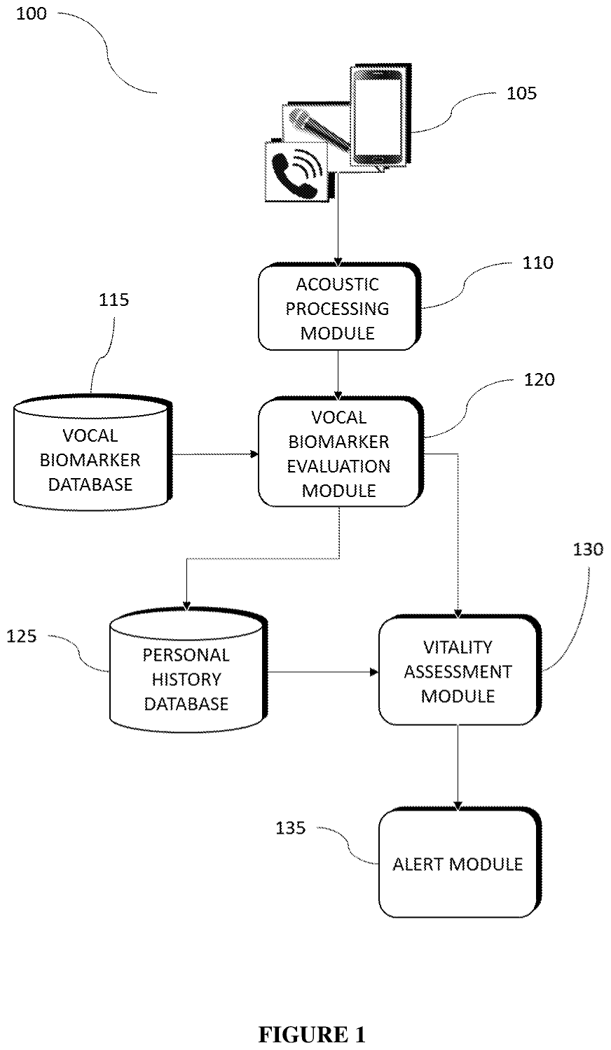

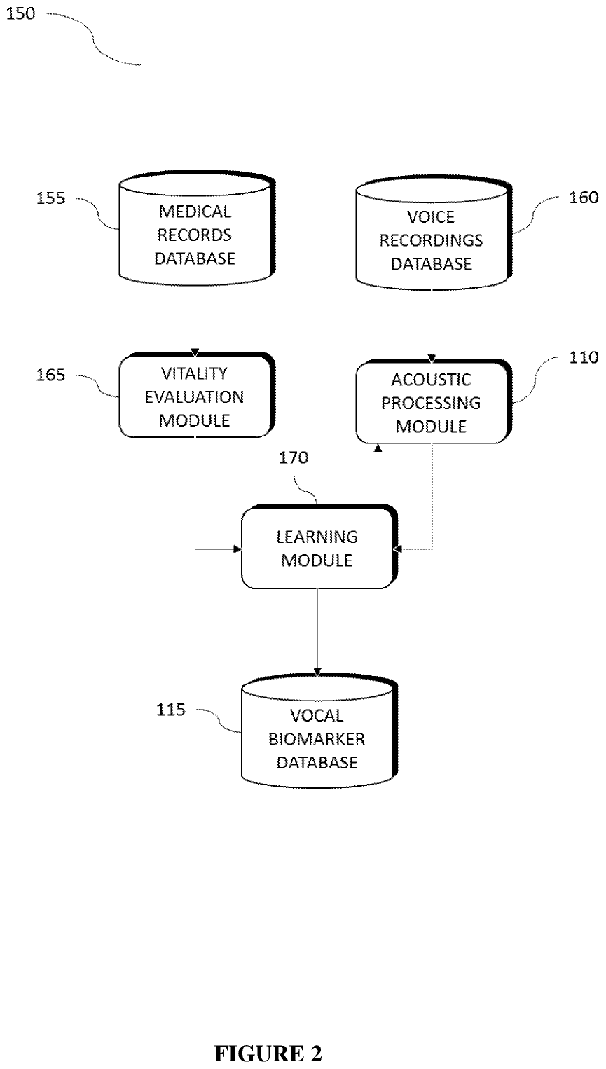

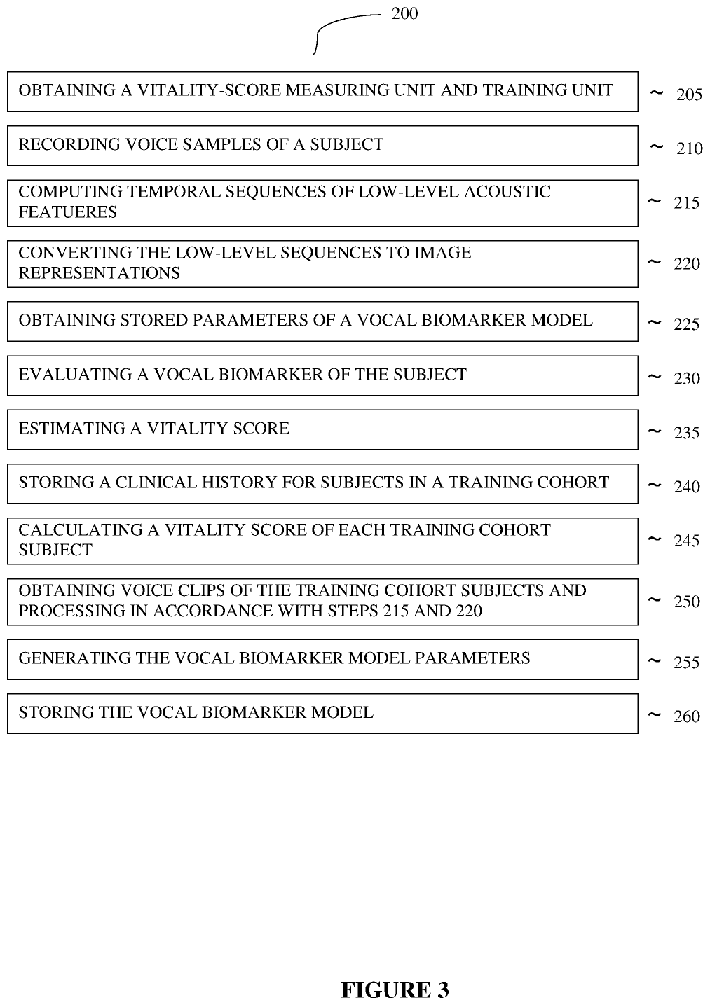

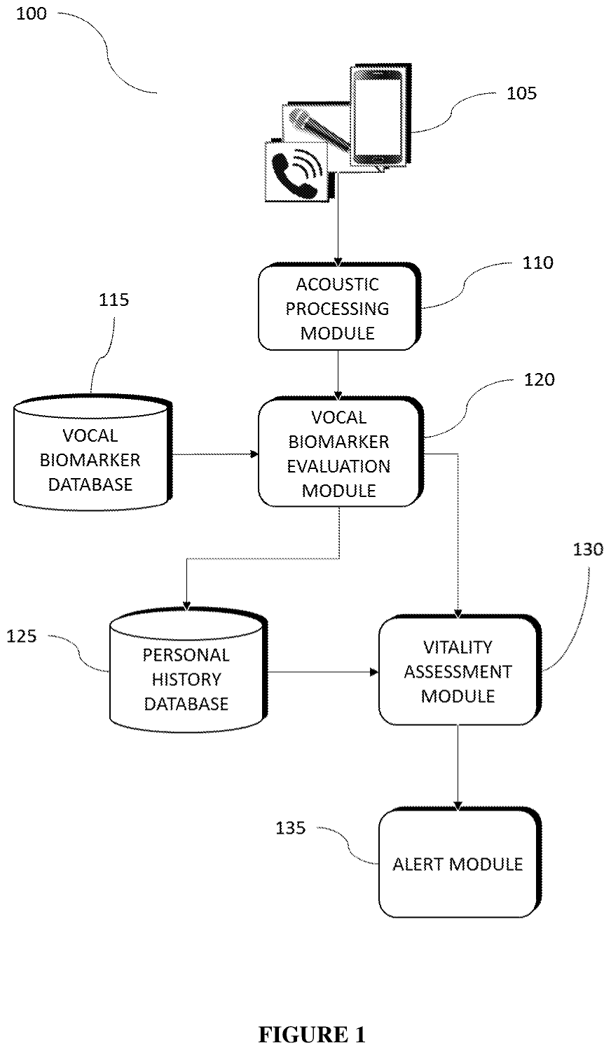

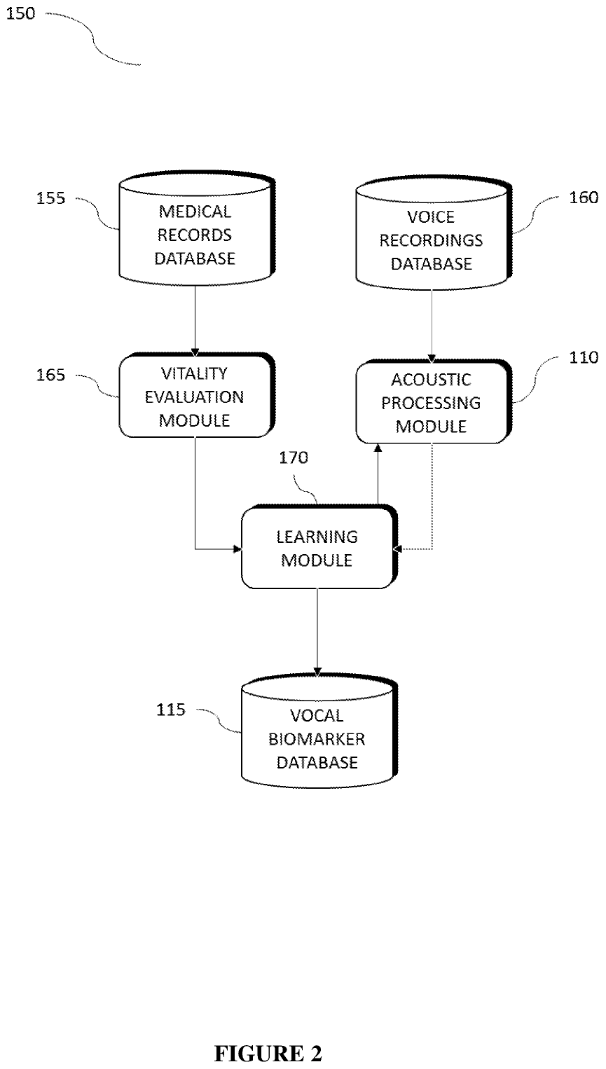

Reference is now made to FIGS. 1 and 2, showing a computer-based system for estimating a vitality score of a subject based on voice, according to some embodiments of the invention. A measuring unit 100 of the system is used to monitor or screen a subject for vitality based on voice and a training unit 150 is used for training measuring unit 100.

### Measuring Unit

One or more recording devices 105 record voice samples of a subject. The recording devices 105 can be any combination of suitable devices, including an audio recorder or telephone call recorder. Recording devices 105 may be placed in personal possession (e.g., worn) or in a home of the subject, and/or in a clinic visited by the subject.

An acoustic processing module 110 computes temporal sequences of a set of low-level acoustic features of each voice sample. Low-level features may include one or more of Mel-frequency cepstral coefficient (MFCC) representations, spectrum representations, pitch and formant measures, chroma and tonal analysis, relative spectral (RASTA) analysis, linear predictive coding (LPC), line spectral pairs (LSP), perceptual linear predictive (PLP) analysis, jitter, shimmer, and loudness.

Acoustic processing module 110 converts the temporal sequences of the set of low-level acoustic features into an image representation, in which one pixel axis represents time and the other axis represents different low-level features in the set. The image representation of the sequence of low-level feature permits employment of image analysis algorithms and deep neural networks for further analysis of voice data.

In some embodiments, acoustic processing module 110 is further configured to calculate high-level features of the image representations. For example, where a learning module 170 (further described herein) of training unit 150 employs a machine learning algorithm, training with high-level feature inputs helps to reduce the volume of processed data to a manageable amount. The high-level acoustic features can include one or more moment analyses comprising analysis of mean, standard deviation, skewness, and kurtosis of the image representations.

A vocal biomarker model file 115 stores parameters of a vocal biomarker model. The vocal biomarker model is constructed by a training unit 150 (further described herein). A vocal biomarker evaluation module 120 evaluates one or more vocal biomarkers of the subject, as a function of the high-level features extracted by acoustic processing module 110. The function used in the evaluation is defined by the vocal biomarker model parameters stored in vocal biomarker model file 115.

A vitality assessment module 130 of measuring unit 100 estimates a vitality score of the subject associated with the voice sample. The estimated vitality score is computed as a function of the evaluated vocal biomarkers.

In some embodiments, a personal history database 125 of measuring unit 100 receives the evaluated vocal biomarkers. Personal history database 125 stores a history of vocal biomarkers of the subject, to which the received vocal biomarkers are added. Vitality assessment module 130 may examine previous vocal biomarkers in the history, in order to improve accuracy of the vitality score.

A display module 135 receives the estimated vitality score from vitality assessment module 130 and displays the vitality score. Display module 135 can be a display, a printout, or any other suitable means of informing medical personnel of the vitality score.

Vitality assessment module 130 can further evaluate the progression and deterioration of diseases of the subject, and estimate risk conditions for acute events. Diseases monitored can include heart diseases such as congestive heart failure, cancer, COPD, diabetes, and other. Additionally, when vitality assessment module 130 finds acute medical events of the subject, it may trigger an alert to medical personnel or caregivers for appropriate intervention.

### Training Unit

A medical records database 155 stores a clinical history of clinical conditions, measurements, and events of subjects in a training cohort. Examples of items in the history include blood pressure measurements, presence of a clinical condition (such as hypertension), occurrence of a heart attack, and occurrence of a stroke.

A voice recordings database 160 stores voice clips of the training cohort subjects. The voice clips may be recorded at a clinic, during visits and/or phone calls of training cohort subjects for treatment. Voice clips of a training cohort subject or the training cohort subject himself may be excluded if there are technical difficulties identifying the subject's voice.

For each training cohort subject, medical records database and/or voice recordings database may be collected over a period of time (e.g., five years).

For each of the training cohort subjects, a vitality evaluation module 165 receives a clinical history from medical records database 155 and calculates a vitality score of the training cohort subject, as a function of the clinical history. (Note that vitality evaluation module 165 calculates a vitality score from clinical data, while vitality assessment module 130 of measuring unit 100 estimates a vitality score from a voice sample.)

Acoustic processing module 110 processes voice clips and extracts image representations or high-level features, as further described herein, from each voice clip.

A learning module 170 generates the parameters of the vocal biomarker model as an optimized association of an aggregation of the 1) vitality scores received from vitality evaluation module 165 with 2) the image representations or high-level features of the voice clips received from acoustic processing module 110.

In some embodiments, learning module 170 employs a deep learning algorithm, in which case the learning module 170 receives and directly processes the image representations to generate the parameters of the vocal biomarker model.

Vocal biomarker model file 115 receives the generated parameters from learning module 170 and stores them.

### Training Examples

In some embodiments, the vitality score of each said training cohort subject, at a time of recording of a voice sample, is defined as a function of an age of the training cohort subject and a time duration elapsed between the time of recording and a said available clinical event.

Clinical events comprise in medical records database may specify a rate of change above a threshold rate in clinical conditions, an emotional state, physiological measurements, or any combination thereof of training cohort subjects.

In some embodiments, a clinical event is death of a subject, hospitalization of said subject, or any combination thereof.

In some embodiments, a vitality score associated with a voice clip of a training cohort subject is binary—either “0” or “1”—and “1” corresponds to “near death,” “near death” defined as when the training cohort subject died within a predefined life-end time interval or the training cohort subject exceeded a life expectancy, at a time the voice clip was recorded. In one implementation, the life-end interval is four years and the life expectancy is 83 years.

In some embodiments, the vocal biomarker model includes parameters for patterns of dynamic behavior between features at a beginning of a voice clip and an end of the voice clip. Such dynamic patterns are generated by acoustic processing module 110. During training, the dynamic patterns are evaluated by learning module 170 and replaced or updated accordingly.

The system of claim 1, wherein said vocal biomarker model is further configured to evaluate, for said subject, the progression and deterioration of diseases and estimate risk conditions for acute events.

In another training example, more than 400 cohort subjects above age 65 with chronic conditions, mainly cardiovascular disease and congestive heart failure, were monitored. The training study revealed a correlation between a future level of glycated hemoglobin (HbA1C) and a vocal score derived by analysis of voice clips of the cohort subjects. A normal HbA1C level in the studied age bracket is 7.0.

The training study found, with more than 80% success, the following correlations between the vocal score of an analyzed voice clip and HbA1C level measured a number months after recording of the voice clip:

| Vocal Score | HbAlC level | No. of months |
|---|---|---|
| 0.89 | 8.8 | 3 |
| 0.97 | 9.5 | 1 |
| 0.14 | 7.0 | 2 |
| 0.04 | 7.3 | 2 |

Thus, a vocal biomarker model for HbA1C level may be developed by training unit 150; and measuring unit 100 can alert medical personnel of energetic deterioration of an organ, months before the next scheduled test for HbA1C would signal the deterioration.

### Measuring and Training Methods

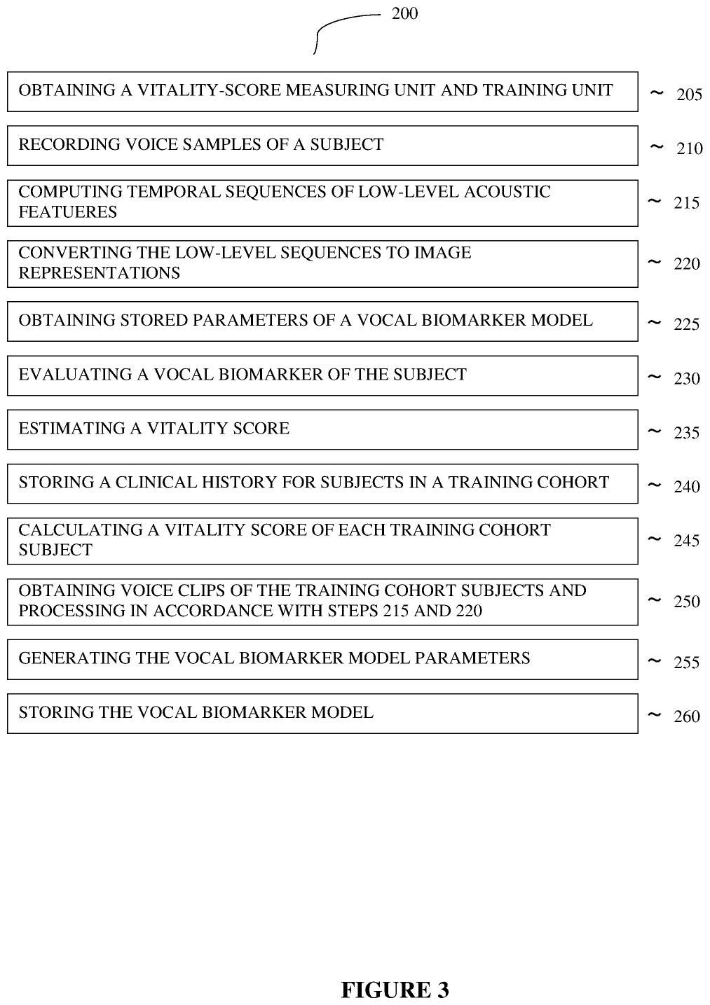

Reference is now made to FIG. 3, showing a computer-based process 200, comprising steps of a measuring method for estimating a vitality score of a subject and steps of a training method for training the measuring method, according to some embodiments of the invention.

Process 200 comprises a step of obtaining a vitality-score measuring unit and training unit 205.

The measuring method comprises steps of
a. recording a voice sample of a subject 210;
b. computing temporal sequences of a set of low-level acoustic features of the voice sample 215;
c. converting the low-level sequences of acoustic features to image representations 220;
d. obtaining stored parameters of a vocal biomarker model 225;
e. evaluating a vocal biomarker of the subject as a function of the image representation, the function defined by the parameters of the vocal biomarker model 230; and
f. estimating a vitality score associated with the voice sample, as a function of the evaluated vocal biomarker 235.

The training method comprises steps of
a. storing a clinical history for subjects in a training cohort 240;
b. calculating a vitality score of each the training cohort subject, as a function of the clinical history of the training cohort subject 245;
c. obtaining voice clips of the training cohort subjects and processing the voice clips in accordance with the steps of computing temporal sequences and of a set of low-level voice features and converting the low-level sequences of acoustic features to image representations 250;
d. generating the parameters of the vocal biomarker model as an optimized association of an aggregation of the vitality scores with the image representations of the training cohort 255; and
e. storing the vocal biomarker model in a vocal biomarker file 260.

1-36. (canceled)

37. A computer-based system, comprising a measuring unit 100 for estimating a vitality score of a subject based on voice and a training unit 150 for training said measuring unit 100, said system comprising one or more processors and non-transitory computer-readable media (CRM), said CRMs storing instructions to said processors for operation of modules of said measuring unit 100 and said training unit 150,
a. said measuring unit 100 comprising
i. one or more recording devices 105, configured to record a voice sample of a subject;
ii. an acoustic processing module 110, configured to
a) compute temporal sequences of a set of low-level acoustic features of said voice sample; and
b) convert said low-level sequences of acoustic features to image representations;
iii. a vocal biomarker model file 115, configured to store parameters of a vocal biomarker model;
iv. a vocal biomarker evaluation module 120, configured to evaluate a vocal biomarker of said subject as a function of said image representation, said function defined by said parameters of said vocal biomarker model; and
v. a vitality assessment module 130, configured to estimate a vitality score associated with said voice sample, as a function of said evaluated vocal biomarker; and
b. said training unit 150 comprising
i. a medical records database 155, comprising a clinical history for subjects in a training cohort;
ii. a vitality evaluation module 165, configured to calculate a vitality score of each said training cohort subject, as a function of said clinical history of said training cohort subject;
iii. a voice recordings database 160, comprising voice clips of said training cohort subjects and their said image representations, extracted by said acoustic processing module 110; and
iv. a learning module 170, configured to generate said parameters of said vocal biomarker model as an optimized association of an aggregation of said vitality scores with said image representations of said training cohort and to store said vocal biomarker model in said vocal biomarker file.

38. The system of claim 37, wherein said set of low-level acoustic features comprises one or more of spectrum representations, Mel-frequency cepstral coefficient (MFCC) representations, pitch and formant measures, chroma and tonal analysis, relative spectral (RASTA) analysis, linear predictive coding (LPC), line spectral pairs (LSP), perceptual linear predictive (PLP) analysis, jitter, shimmer, loudness, and any combination thereof.

39. The system of claim 37, wherein said learning module employs a machine learning algorithm and generates said vocal biomarker model as a function of high-level features of said image representation; said acoustic processing module further configured to compute said high-level features, further said learning module employs a deep learning algorithm that directly processes said image representations to generate said vocal biomarker model

40. The system of claim 39, wherein said high-level features comprise moment-analysis measurements of said low-level features, said moment analyses comprising analysis of mean, standard deviation, skewness, and kurtosis of said image representations.

41. The system of claim 37, wherein said vitality score of each said training cohort subject, at a time of recording of said voice sample, is defined as a function of clinical conditions, an emotional state, physiological measurements, or any combination thereof of said training cohort subjects.

42. The system of claim 41, wherein said vitality score is further
a. a function of an age of said training cohort subject and a time duration elapsed between the time of recording and one or more available clinical events selected from a group comprising death of said subject, hospitalization of said subject, a measurement of glycated hemoglobin (HbA1c) level, or any combination thereof.
b. binary—either “0” or “1”—and “1” corresponds to “near death,” “near death” defined as when said training cohort subject died within a predefined life-end time interval of four years or said training cohort subject exceeded a life expectancy of eighty-three years, at a time said voice clip was recorded.

43. The system of claim 42, wherein said vitality scores associated with said voice clips correspond to future HbA1c levels.

44. The system of claim 37, wherein said vocal biomarker model includes parameters for patterns of dynamic behavior between said features at a beginning of a said voice clip and an end of said voice clip, said vocal biomarker model configured to evaluate, for said subject, the progression and deterioration of one or more diseases, inter alia, congestive heart failure, and estimate risk conditions for acute events and to issue an alert for acute medical events of said subject.

45. The system of claim 37, further comprising a personal history database configured to receive and store said evaluated vocal biomarkers to a history of said vocal biomarkers of said subject and wherein said vitality score is further a function of said history.

46. The system of claim 45, wherein said voice clips and clinical events of one or more of said subjects are collected over a period of time.

47. A computer-based method, comprising a measuring method 200 for estimating a vitality score of a subject based on voice and a training method 250 for training said measuring method, comprising a step of obtaining a system of claim 1 205, and further steps
a. of said measuring method 200 comprising:
i. recording a voice sample of a subject 210;
ii. computing temporal sequences of a set of low-level acoustic features of said voice sample 215;
iii. converting said low-level sequences of acoustic features to image representations 220;
ii. obtaining stored parameters of a vocal biomarker model 225;
iii. evaluating a vocal biomarker of said subject as a function of said image representation, said function defined by said parameters of said vocal biomarker model 230; and
iv. estimating a vitality score associated with said voice sample, as a function of said evaluated vocal biomarker 235; and
b. of said training method 250 comprising:
i. storing a clinical history for subjects in a training cohort 240;
ii. calculating a vitality score of each said training cohort subject, as a function of said clinical history of said training cohort subject 245;
iii. obtaining voice clips of said training cohort subjects and processing said voice clips in accordance with said steps of computing temporal sequences and of a set of low-level voice features and converting said low-level sequences of acoustic features to image representations 250;
iv. generating said parameters of said vocal biomarker model as an optimized association of an aggregation of said vitality scores with said image representations of said training cohort 255; and
v. storing said vocal biomarker model in a vocal biomarker file 260.

48. The method of claim 47, wherein said set of low-level acoustic features comprises one or more of spectrum representations, Mel-frequency cepstral coefficient (MFCC) representations, pitch and formant measures, chroma and tonal analysis, relative spectral (RASTA) analysis, linear predictive coding (LPC), line spectral pairs (LSP), perceptual linear predictive (PLP) analysis, jitter, shimmer, loudness, and any combination thereof.

49. The method of claim 47, further comprising steps of
a. computing high-level features of said image representation and employing a machine-learning algorithm to generate said vocal biomarker model as a function of said high-level features,
b. employing a deep learning algorithm that directly processes said image representations to generate said vocal biomarker model,
c. receiving and storing said evaluated vocal biomarkers to a history of said vocal biomarkers of said subject, wherein said vitality score is further a function of said history,
d. evaluating, for said subject, the progression and deterioration of one or more diseases, inter alia congestive heart failure, and estimating risk conditions for acute events.

50. The method of claim 49, wherein said high-level features comprise moment-analysis measurements of said low-level features, said moment analyses comprising analysis of mean, standard deviation, skewness, and kurtosis of said image representations.

51. The method of claim 47, wherein said vitality score of each said training cohort subject, at a time of recording of said voice sample, is defined as a function of clinical conditions, an emotional state, physiological measurements, or any combination thereof of said training cohort subjects.

52. The method of claim 51, wherein said vitality score is further
a. a function of an age of said training cohort subject and a time duration elapsed between the time of recording and one or more available clinical events, selected from a group comprising death of said subject, hospitalization of said subject, a measurement of glycated hemoglobin (HbA1c) level, or any combination thereof
b. a binary—either “0” or “1”—and “1” corresponds to “near death,” “near death” defined as when said training cohort subject died within a predefined life-end time interval of four years or said training cohort subject exceeded a life expectancy of eighty-three, at a time said voice clip was recorded.

53. The method of claim 52, wherein said vitality scores associated with said voice clips correspond to future HbA1c levels.

54. The method of claim 47, wherein said vocal biomarker model includes parameters for patterns of dynamic behavior between said features at a beginning of a said voice clip and an end of said voice clip.

55. The method of claim 49, wherein said voice clips and clinical events of one or more of said subjects are collected over a period of time.

56. The method of claim 55, further comprising a step of issuing an alert for acute medical events of said subject.
